(19) United States
(12) Patent Application Publication
(10) Pub. No.: US 2024/0182831 A1
(43) Pub. Date: Jun. 6, 2024

(54) A WELL INSERT AND A DEVICE FOR 3D CELL CULTURE AND IN VITRO TISSUE MODEL

(71) Applicant: AGENCY FOR SCIENCE, TECHNOLOGY AND RESEARCH, Singapore (SG)

(72) Inventors: Andrea PAVES, Singapore (SG); Giulia ADRIANI, Singapore (SG)

(73) Assignee: AGENCY FOR SCIENCE, TECHNOLOGY AND RESEARCH, Singapore (SG)

(21) Appl. No.: 18/550,256

(22) PCT Filed: Mar. 11, 2022
(86) PCT No.: PCT/SG2022/095001

§ 371 Date: Sep. 12, 2023

(30) Foreign Application Priority Data
Mar. 16, 2021 (SG) 10202102643V

(51) Int. Cl. C12M 1/32; C12M 1/00; C12M 1/12

(52) U.S. Cl. CPC C12M 23/12; C12M 23/34; C12M 25/04

(57) ABSTRACT

Multi-well plate compatible device or insert for three-dimensional (3D) cell cultures, use and kit thereof are provided. The device includes outer and inner walls. the inner wall defining a volume and is located within the outer wall, forming a cavity therebetween. It also includes a base configured to position a cell culture sample below the volume within the inner wall and one or more partitions connected to the base. connecting the inner wall to the outer wall and segmenting the cavity into a plurality of voids. Openings in surfaces forming the voids allow fluid flow from a first void. below the volume within the inner wall to a second void, the fluid flow configured to interact with the cell culture sample when flowing through a sample region within the inner wall. The sample region has a depth defined by a height of the inner wall greater than a length defined by an inner distance across the volume within the inner wall.

### PRIORITY CLAIM

This application claims priority from Singapore Patent Application No. 10202102643V filed on 16 Mar. 2021.

### TECHNICAL FIELD

The present invention generally relates to cell culture well plates, and more particularly relates to a well insert for three-dimensional cell cultures, such as a cell culture well insert for a multi-well cell plate.

### BACKGROUND OF THE DISCLOSURE

In precision oncology, omics, such as next-generation sequencing, mRNA-sequencing, ChIP-sequencing, and mass spectrometry, determine the patient-specific tumour profile to identify mutations that could be treated by existing anticancer drugs. Despite the advances in personalized therapy, cancer remains incredibly challenging because not all tumours carry a mutation that can be targeted with existing drugs. In addition, existing omics data for each tumour type are limited and tumours are highly heterogeneous meaning they could have mutations in their metastasis that are not present in the primary tumour and this can lead to different responses to the same therapy. This further leads to the fact that even identified biomarkers do not fully react to a therapy targeting those biomarkers or developed towards those biomarkers.

The current method of performing pre-clinical drug testing on tumour biopsies is using mouse patient-derived-xenograft (PDX) models. Tumour cells are implanted into immunodeficient mice to follow tumour progression during treatment. Although PDXs can provide some response prediction, they have limitations restricting their practical applications in clinical settings. For example, PDX has low engraftment and is both costly and time consuming as the time to get results could range between two to twelve months. These drawbacks make PDX models incompatible with the speed and high-throughput required for precision medicine.

Thus, there is a need for devices to enable testing patient-specific tumour sensitivity to anticancer compounds before clinical treatment administration which simplifies testing therapeutic strategies on patient-derived tumour organoids yet retains tumour complexity and allows for a rapid drug screening. There is also a need to build 3D in vitro models able to mimic in vivo complexity and to provide tools to culture organoids in a physiological relevant environment. Furthermore, other desirable features and characteristics will become apparent from the subsequent detailed description and the appended claims, taken in conjunction with the accompanying drawings and this background of the disclosure.

### SUMMARY

According to at least one aspect of the present embodiments, a multi-well plate compatible cell culture device, such as an insert for a multi-well cell plate, is provided. The cell culture well device includes an outer wall, an inner wall, a base, and one or more partitions. The inner wall is located within the outer wall and forms a cavity therebetween. The inner wall also defines a volume therewithin. The base is connected to a bottom of the outer wall and a bottom of the inner wall and is configured to position a cell culture sample below the volume within the inner wall. The one or more partitions are connected to the base and connect the inner wall to the outer wall, the one or more partitions segmenting the cavity into a plurality of voids. Openings in surfaces forming the voids allow fluid flow from a first one of the voids and below the volume within the inner wall to a second one of the voids, the fluid flow configured to interact with the cell culture sample when flowing through a sample region within the inner wall. The sample region has a depth defined by a height of the inner wall greater than a length defined by an inner distance across the volume defined within the inner wall.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to illustrate various embodiments and to explain various principles and advantages in accordance with present embodiments.

FIG. 1 illustrates clinical workflow and details of a cell culture well insert in accordance with the present embodiments.

FIG. 2, comprising FIGS. 2A to 2H, depicts details of the cell culture well insert in accordance with the present embodiments, wherein FIG. 2A is a side elevation planar view, FIG. 2B is a bottom planar view, FIG. 2C depicts a top planar view looking into the cell culture well insert, FIG. 2D is an angled top perspective view looking into the cell culture well insert, FIG. 2E is an angled bottom view showing a hydrogel encased cell culture sample, FIG. 2F is a cutaway angled top view showing the hydrogel encased cell culture sample, FIG. 2G is a cutaway elevation view showing the hydrogel encased cell culture sample, and FIG. 2H is the cutaway elevation view of FIG. 2G showing the cell culture well insert during use.

FIG. 3, comprising FIGS. 3A, 3B and 3C, depicts photographs of a cell culture well insert in accordance with the present embodiments, wherein FIG. 3A depicts a top, side perspective view and a bottom angled perspective view of cell culture well inserts, FIG. 3B depicts a top planar view, and FIG. 3C depicts a bottom planar view.

FIG. 4, comprising FIGS. 4A and 4B, depicts photographs of bottom, side perspective views of two designs for a cell culture well insert in accordance with the present embodiments, wherein FIG. 4A depicts a net design and FIG. 4B depicts a pillar design.

FIG. 5, comprising FIGS. 5A to 5E, depicts use of a cell culture well insert in a multi-well cell culture plate in accordance with the present embodiments, wherein FIG. 5A depicts insertion of the cell culture well insert into a well of the multi-well cell culture plate, FIG. 5B depicts a top perspective view of the cell culture well insert seated in the well of the multi-well cell culture plate, FIG. 5C depicts a top perspective view of two cell culture well inserts seated in the multi-well cell culture plate, FIG. 5D depicts a bottom perspective view of the cell culture well insert seated in the well of the clear multi-well cell culture plate, and FIG. 5E depicts a top planar view of the cell culture well insert seated in the well of the multi-well cell culture plate.

FIG. 6, comprising FIGS. 6A and 6B, depicts testing protocols for use with the cell culture well insert in accordance with the present embodiments, wherein FIG. 6A depicts an illustration of solid tumour vascularization of an organoid from a patient biopsy, and FIG. 6B depicts an illustration of developing a secondary tumour model to screen for treatment.

FIG. 7, comprising FIGS. 7A to 7C, depicts test results of the efficacy of the cell culture well insert in accordance with the present embodiments, wherein FIG. 7A depicts a fluorescent microscopy image of a bottom view of the cell culture well insert, FIG. 7B depicts a fluorescent microscopy image of results of tests in accordance with FIG. 6A, and FIG. 7C depicts a fluorescent microscopy image of results of tests in accordance with FIG. 6B.

FIG. 8, comprising FIGS. 8A and 8B, depicts fluorescence microscopy images of hepatocellular carcinoma cell aggregates expressing green fluorescent protein (GFP) surrounded by a collagen hydrogel, wherein FIG. 8A depicts the hepatocellular carcinoma cell aggregates with embedded endothelial cells and FIG. 8B depicts hepatocellular carcinoma cell aggregates with necrotic core typical of hepatocellular carcinoma cell aggregates.

FIG. 9, comprising FIGS. 9A and 9C, depicts details of a pillar design for a cell culture well insert in accordance with the present embodiments, wherein FIG. 9A depicts a bottom view angle, FIG. 9B depicts a bottom/side view angle, and FIG. 9C depicts a side bottom view angle.

FIG. 10, comprising FIGS. 10A to 10F, depicts details of a net design for a cell culture well insert in accordance with the present embodiments, wherein FIG. 10A depicts a first bottom view angle, FIG. 10B depicts a second bottom view angle, FIG. 10C depicts a first bottom/side view angle, FIG. 10D depicts a second bottom/side view angle, FIG. 10E depicts cross-section angled view, and FIG. 10F depicts a cross-section planar view.

FIG. 11, comprising FIGS. 11A and 11B, depicts an illustration of a half-wall design for a cell culture well insert in accordance with the present embodiments, wherein FIG. 11A depict s a top/side view angle and FIG. 11B depicts a cross-sectional planar view.

FIG. 12 depicts an illustration of a half-deep design for a cell culture well insert in accordance with the present embodiments.

FIG. 13 depicts a quarter-well design for a cell culture well insert in accordance with the present embodiments.

FIG. 14 depicts confocal microscopy images of in situ immunofluorescent staining of a liver organoid cultured in collagen hydrogel in a cell culture well insert in accordance with the present embodiments.

FIG. 15, comprising FIGS. 15A and 15B, depicts confocal microscopy images of vascularized tumors in a half-wall design of a cell culture well insert in accordance with the present embodiments, wherein FIG. 15A depicts an image of results of tests in accordance with the vascularized process depicted in FIG. 6A and FIG. 15B depicts an image of a bottom view of the cell culture well insert including the results of the tests of FIG. 15A.

FIG. 16, comprising FIGS. 16A and 16B, depicts confocal microscopy images of vascularized tumors in a “pillar” design of a cell culture well insert in accordance with the present embodiments, wherein FIG. 16A depicts an image of results of tests in accordance with the vascularized process depicted in FIG. 6A and FIG. 16B depicts an image of a bottom view of the cell culture well insert including the results of the tests of FIG. 16A.

FIG. 17, comprising FIGS. 17A and 17B, depicts a process for removing a fresh (not frozen) sample from a cell culture well insert in accordance with the present embodiments, wherein FIG. 17A is a photograph depicting a stage immediately after the sample is pushed from the cell culture well insert and FIG. 17B is a photograph depicting a later stage where the sample is collected in a petri dish.

FIG. 18, comprising FIGS. 18A to 18D, depicts a process for removing a frozen sample from a cell culture well insert in accordance with the present embodiments, wherein FIG. 18A is a photograph depicting a stage where a bottom laminate is removed from the cell culture well insert, FIG. 18B is a photograph from above depicting a stage where the frozen sample is pushed out from the cell culture well insert and collected in a petri dish, FIG. 18C is a photograph toward a bottom of the cell culture well insert depicting the stage of FIG. 18B, and FIG. 18D is a photograph depicting a later stage where the frozen sample is placed in a “cassette” for histology sample preparation.

Skilled artisans will appreciate that elements in the figures are illustrated for simplicity and clarity and have not necessarily been depicted to scale.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. It is the intent of present embodiments to present a cell culture well insert. The well insert in accordance with present embodiments can be used in any cell culture well plate and can be dimensioned to the size of the individual well.

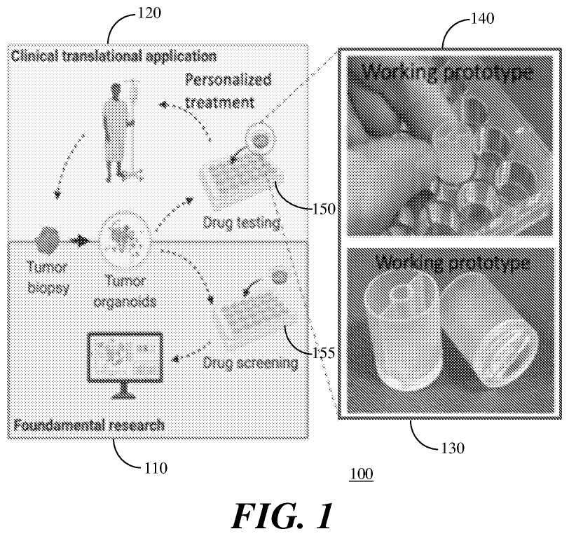

Referring to FIG. 1, an illustration 100 depicts a clinical workflow including fundamental research 110 and clinical translational application 120 of the research utilizing drug testing with a cell culture well insert 130 in a multi-well cell culture well plate 140 in accordance with the present embodiments. The cell culture well insert 130 is used to grow patient biopsy ex vivo for drug testing 150 and drug screening 155.

The cell culture well insert 130 in accordance with the present embodiments is a product for ex vivo testing of, for example, a panel of anticancer compounds in order to define drug sensitivity and resistance of vascularized patient-derived tumour organoids cultured in a three-dimensional (3D) extracellular matrix. Ex vivo data can be combined with information from molecular profiling to provide a comprehensive picture of tumour response, thereby advantageously helping to identify the most appropriate therapy for each patient. Moreover, patient samples can be treated with libraries of compounds and combinations of compounds approved by the United States Food and Drug Administration (FDA) to screen for anticancer activity for drug repurposing.

The cell culture well insert 130 in accordance with the present embodiments advantageously enables fast and high throughput testing of patient-derived cells as shown in the clinical workflow 120. Results can be obtained within a few days with low cell number requirement. The implementation of ex vivo sensitivity tests as a routine in clinical practice can help clinicians in the decision-making step, beneficially opening a new era for successful precision medicine in cancer treatment. Thus, the cell culture well insert 130 in accordance with the present embodiments, along with appropriate validation, can become a gold standard to test therapeutic strategies on patient-derived tumour organoids as the cell culture well insert 130 advantageously retains tumour complexity while allowing for rapid drug screening.

Three-dimensional (3D) cell culture is gaining importance exponentially in the cell biology field due to the need for researchers to study cell phenomena in a more physiological culture system, compared to two-dimensional (2D) surfaces. Several disease models and drug testing platforms are implementing a 3D cell culture to mimic the microenvironment that cells sense in vivo. However, there's still a need to develop improved technologies enabling a reliable yet simple 3D cell culture taking which provides high-throughput, cost effectiveness and operational simplicity.

In particular, organoids and spheroids need to be surrounded by an extracellular matrix-like environment and to be co-cultured with supporting cells to mimic and retain characteristics of human tissue. Existing methods to grow spheroids/organoids in a multi-well plate format present many limitations such as a lack of pressure and chemical gradients that are fundamental for many biological mechanisms such as cell migration, homing, and cell development. Further, today there is an urgent need for high-throughput assays to speed up drug screening and drug development processes. To meet this need, the cell culture well insert in accordance with the present embodiments. The cell culture well insert is a tool that is very simple to implement and use and which can be up-scaled as required.

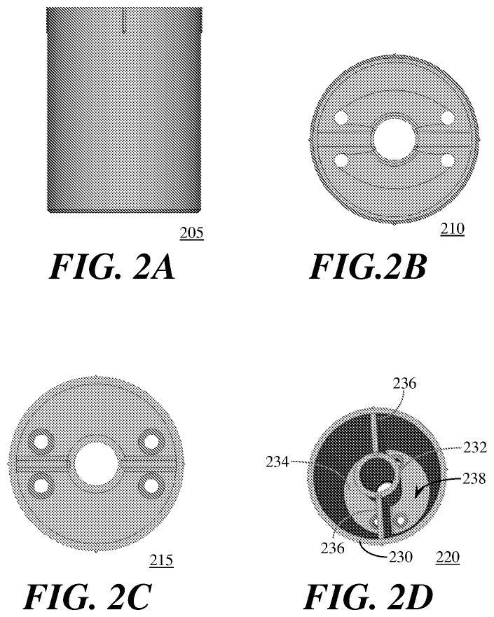

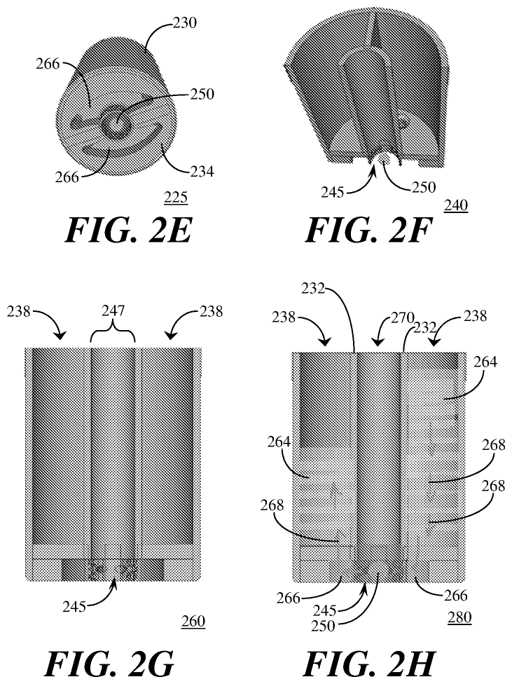

FIGS. 2A to 2C depict details of the cell culture well insert in accordance with the present embodiments in a side elevation planar view 205 (FIG. 2A), a bottom planar view 210 (FIG. 2B), and a top planar view 215 (FIG. 2C) looking into the cell culture well insert. FIG. 2D depicts an angled top perspective view 220 looking into the cell culture well insert and FIG. 2E depicts an angled bottom view 225.

The cell culture well insert in accordance with the present embodiments, as seen in FIGS. 2D and 2E, includes a plastic cylinder forming an outer wall 230, an inner wall 232 (e.g., a cylinder or other three-dimensional shape) defining a volume within the inner wall 232, a base 234 connected to the bottom of the inner wall 232 and the outer wall 230, and partitions 236 connected to the base 234 and connecting the inner wall 232 to the outer wall 230. The partitions 236 segment a cavity formed between the inner wall 232 and the outer wall 230 into a plurality of voids 238. In this manner, the cell culture well insert pictured in FIGS. 2A to 2E includes two reservoirs (the two voids 238) thus including three chambers (the two reservoirs and the volume within the inner wall 232). Referring to FIGS. 2F and 2G, a cutaway angled top view 240 and a cutaway side elevation planar view 260 depict a sample region 245 defined at a bottom of the volume formed by the inner wall 232. The sample region 245 has a depth defined by a height of the inner wall 232 and the depth is greater than a length defined by an inner distance 247 across the volume defined within the inner wall 232. Using standard cell culture pipettes and tips, a user can add a hydrogel encased cell culture sample (a hydrogel 250 with a sample of cells, organoids, tissue or other organic material embedded therein) in the sample region 245. The hydrogel 250 can host single cells or organoids and spheroids, according to or as required by the desired experimental protocol. The two reservoirs 238 can be filled with cells as well to perform multi-culture assays, or the reservoirs 238 can be filled with culture media and various soluble factors to hydrate the hydrogel and support the 3D cell culture.

While it has been mentioned that the outer wall 230, the inner wall 232, the base 234, and the partitions 236 can be formed of plastic, any polymer or similar biocompatible material for mass production of the cell culture well insert in accordance with the present embodiments may be used, such as polymethylpentene (PMP), polystyrene, or polycarbonate.

A membrane may also be used in accordance with the present embodiments to seal the entire bottom of the cell culture well insert for easy retrieval of the cell culture from the sample region 245 without leaking. The membrane may be glued to flat bottom surfaces by, for example, an ultraviolet (UV) curable glue, and may be non-permeable or permeable depending on the membrane material used. Thus, the permeability of the membrane is a function of the material selected, such as PMP which is permeable to oxygen. Sample regions in conventional devices typically have a length which is greater than its depth. When a user attempts to access the hydrogel in the sample region by separating the device from its ‘coverslip’ such as the membrane discussed herein, the gel can stick to either the coverslip or the device, or partially to both. The root cause of this problem is that the surface area of the hydrogel that is in contact with the coverslip is similar to the surface area of the hydrogel that is in contact with the device. The hydrogel sticking issue during the removal process makes it difficult to retrieve the hydrogel and its contents (i.e., the sample) intact for downstream analysis. However, in accordance with the present embodiments, the depth of the sample region 245 is greater than its length 247 as the hydrogel 250 is located within a vertical column as opposed to a horizontal channel. The present design of the cell culture well insert advantageously enables the hydrogel 250 including an organoid, a biopsy, a vasculature, or a similar sample to be retained in and successfully removed from the sample region 245, as there is more surface area in the column formed by the inner wall 232 that is in contact with the gel than the well bottom/membrane surface. Thus, successful removal of the hydrogel 250 from the cell culture well insert in accordance with the present embodiments advantageously enables samples of cultured cells or biopsies to be retrieved with their spatial organization intact for downstream analysis by cryosectioning, histology, digital spatial profiling, or similar processing.

Referring to FIG. 2H, a cutaway elevation view 280 depicts the cell culture well insert during use. In accordance with the present embodiments, the hydrogel 250, due to its viscosity, remains trapped in a central region of the insert, i.e., the sample region 245, by surface tension thanks to the presence of pillars or a “micro-net” feature or half walls as discussed hereinafter. After the hydrogel 250 is added and polymerized (a first step in the application protocol), a cell culture medium 264 can be added in the two reservoirs 238, called the cell media reservoirs. At the bottom of the the cell culture well insert, the reservoirs 238 are connected to “half-moon” shaped channels 266 (the “half-moon” shape better seen in FIG. 2E) through a hole allowing the media 264 to flow (as indicated by arrows 268) in the dedicated fluidic section. The flow results from pressure gradients in the two reservoirs 238 created in response to a pressure generated from a difference in height of the cell culture media 264 liquid volume in the chambers and is transferred to the channels 266 resulting in a lateral force to the hydrogel volume. The media 264 can then cross the hydrogel region by going through the space in between the pillars or the holes in the “micro-net” design or the half walls.

Pressure gradients can be generated thanks to the presence of two distinct medium reservoirs 238 with the hydrogel 262 in the middle allowing perfusion of the hydrogel 262 with the cell culture medium 264. While not shown in FIG. 2H, a central chamber 270 consisting of the volume within the inner wall 232 can also be used to add a volume of media on top of the hydrogel 250. The pressure generated from the height of the cell culture media liquid volume in the central chamber 270 is directly applied to the sample region 245 when media is added to the central chamber 270. Chemical gradients of diffusible factors such as cytokines, growth factors, hormones, and antibodies are also supported. Importantly, the cell culture well insert can be extracted from the well in accordance with the present embodiments to retrieve cells and supernatants for further biological analysis.

Basically, the pressure gradient flows the culture media around the tissue/organoids 250 embedded in the hydrogel 262 in the central location of the insert, i.e., the sample region 245. The pressure gradient can be controlled across the hydrogel region 250 by controlling the amount of liquid volume added in the reservoirs 238 and/or the central chamber 270. Chemical gradients and fluid flow can then be generated in accordance with the present embodiments from left to right, right to left or from top to side. It is to be appreciated that the examples disclosed herein are non-limiting examples of embodiments which fulfil the stated criteria. It is further understood that, for example, the fluid flowing through the sample anchored in the cell culture insert disclosed herein is moved by pressure gradient/difference in volume in the reservoirs 238 and/or the central chamber 270.

In order to achieve a gradient of certain diffusible factors, the cell culture media 264 added to different reservoirs 238 may include different concentrations of diffusible factors. The diffusible factors will then move gradually down the gradient through openings at the bottom, resulting in the cell culture in the inner chamber exposed to different concentrations of the diffusible factors in different directions. Those skilled artisans will realize that one can adjust the concentration gradient and use pure diffusion mechanism between chambers or alternatively adjust the volume difference between the reservoirs 238 to create interstitial fluid flow. In the first case, adding a chemical compound or antibody in one of the reservoirs 238, or in the central chamber formed by the inner wall 232, will diffuse to the direction of less concentration if fluid is at the same level. In this case, any time-dependent diffusion curve would be a function of the composition of the hydrogel 262. A vascularized hydrogel will present a vasculature where drugs/molecules/antibodies can easily flow into the formed “pipe” of low resistance. Alternatively, where an empty hydrogel at a high concentration of collagen will represent a higher resistance and obstacle for diffusion. In accordance with the present embodiments, it has been seen that antibodies can diffuse into the hydrogel over a twelve-hour incubation time. For volume difference between the reservoirs 238 to create interstitial fluid flow to create a pressure difference to drive gradients actively, based on the hydrogel used, the reservoirs 238 will reach equilibrium after twenty-four hours if no media change is performed.

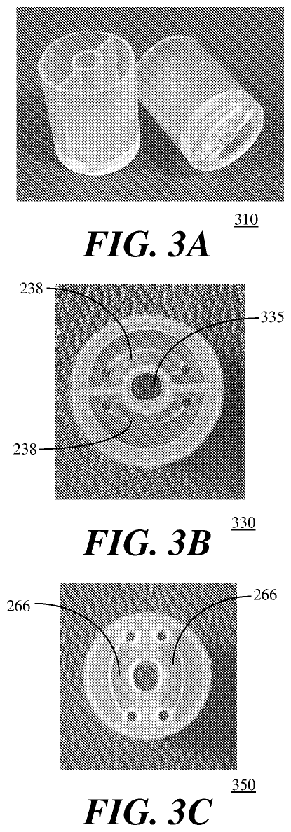

FIGS. 3A to 3C depict photographs 310, 330, 350, respectively, of a cell culture well insert in accordance with the present embodiments. The photograph 310 depicts a top, side perspective view and a bottom angled perspective view of cell culture well inserts, while the photograph 330 depicts a top planar view of the cell culture well insert. In the photograph 330, the volume formed by the inner wall 232 provides an access port 335 for hydrogel deposition and organoid seeding. The photograph 350 depicts a bottom planar view of the cell culture well insert where the lateral “half-moon” shaped channels 266 are seen.

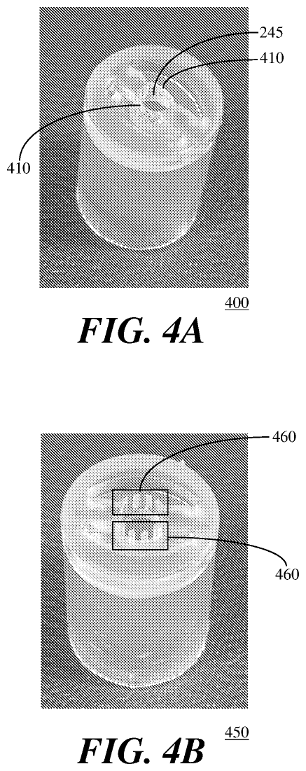

FIGS. 4A and 4B depict photographs 400, 450 of bottom, side perspective views of two designs for a cell culture well insert in accordance with the present embodiments. The two designs differ in design of tissue traps in the sample region 245 where the photograph 400 depicts a net structure 410 in the sample region 245 and the photograph 450 depicts a pillar structure 460 in the sample region 245.

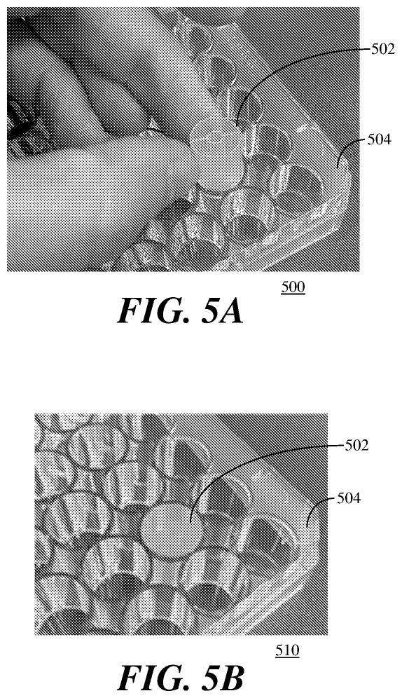

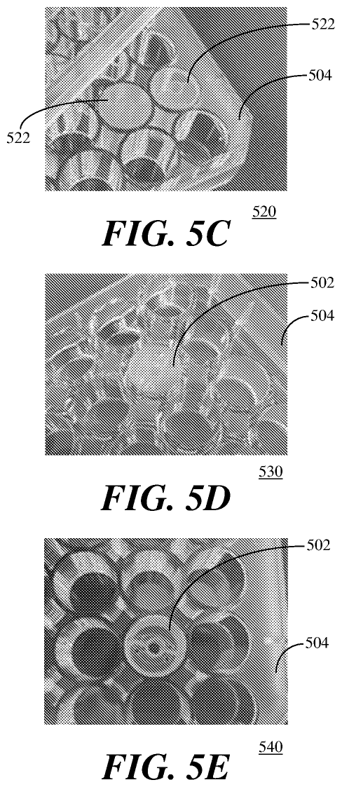

The cell culture well insert in accordance with the present embodiments is designed as disposable lab consumable plastic piece and is dimensioned to fit snugly inside a well of a multi-well cell culture plate. For example, the cell culture well insert in accordance with the present embodiments advantageously fits into a well of a standard 24 or 48 multi-well plate to create a multi-chamber environment for 3D cell culture. Sterile cell culture well inserts can be placed inside the wells by a press fit that ensures tight fastening required to perform cell culture within the different chambers. FIG. 5A depicts a photograph 500 of insertion of a cell culture well insert 502 into a well of a multi-well cell culture plate 504. FIG. 5B is a photograph 510 depicting a top perspective view of the cell culture well insert seated in the well of the multi-well cell culture plate, while FIG. 5C is a photograph 520 that depicts a top perspective view of two cell culture well inserts 522 seated in the multi-well cell culture plate 504. FIG. 5D is a photograph 530 depicting a bottom perspective view of the cell culture well insert 502 seated in the well of the clear multi-well cell culture plate 504, and FIG. 5E is a photograph 540 depicting a top planar view of the cell culture well insert 502 seated in the well of the multi-well cell culture plate 504.

The bottom of the well in the multi-well cell culture plate 504 will act as the bottom surface of the cell culture well insert 502. Alternatively, the cell culture well insert 502 can be used with a bottom laminate in accordance with an aspect of the present embodiment. Usually, commercially available multi-well cell culture plates have a thick bottom surface, sometimes more than one millimeter thick. A thick bottom surface will interfere with high-resolution microscopy, so a cell culture well insert in accordance with the present embodiments can be used with a bottom laminate as a standalone cell culture device instead of an insert for a multi-well cell culture plate for those instances when better microscopy performance is desired, since the laminate can be provided as small as only ˜100 um in thickness. Instead of use as a standalone device, the laminated cell culture well insert can be used with a dedicated holder such as a bottomless well or multi-well plate.

While an unlaminated cell culture well insert can be separated from a well of a multi-well cell culture plate without leaving the hydrogel stuck on the well's bottom surface, the laminated variation will incorporate a void on the external surface of the cell culture well insert close to the bottom of the insert structure that enables easy removal of the laminate to retain the hydrogel in the central gel column, offering both leak-proof culture and easy retrieval. The void will allow a user to use a tweezer to pinch and peel the laminate off the cell culture well insert. The adhesive used to attach the laminate should be strong enough to withstand the hydrostatic pressure and the humidity without delaminating while, at the same time, be weak enough to be easily pulled off by hand with a tweezer.

Advantages of using the cell culture well insert in accordance with the present embodiments in testing drugs on ovarian cancer (OC) biopsies are shown as an example. The decision was made to perform testing on ovarian cancer, since there are limited treatment options available beyond first- and second-line treatment. In addition, ovarian cancer is the fifth most common cancer in women in Singapore and the fifth most common cause of cancer death in Singapore, with an urgent need for efficacious therapeutic approaches to achieve long-term clinical remission. The scientific community's rising demand for shifting from 2D to 3D technologies is further pushing the growth of this market.

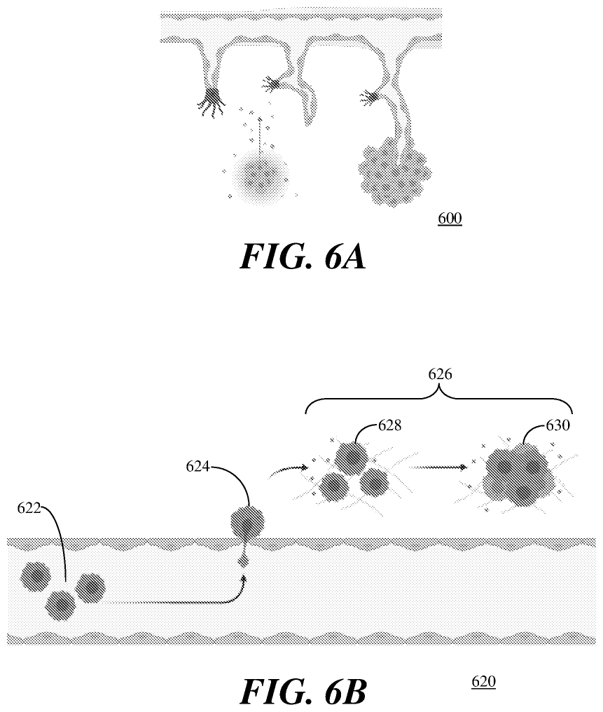

The capabilities of the cell culture well insert in accordance with the present embodiments which allows the 3D culture of tumour biopsies and organoids was tested. In particular, culture patient-derived ovarian carcinoma organoids were used to screen for drug libraries in order to identify the best therapeutic regimen specific for each patient in a clinically-relevant time frame. Referring to FIG. 6A and 6B, illustrations 600, 620 depict two main aims of the study.

The first aim of developing an organoid model of a solid tumour vascularization from a patient biopsy is depicted in the illustration 600 which shows a process of tumour vascularization of cancer cells. Fresh biopsies maintain critical genetic and phenotypic features enabling their use in drug screening and immunotherapy to identify each patient's best therapeutic regimen. This part of the project allowed screening and comparing different chemotherapy, anti-angiogenesis, and immunotherapy approaches and their combination to predict an individual patient's clinical response and help the clinicians choose the more efficacious treatment for each patient.

The second aim of developing a secondary tumour model to screen for treatment is depicted in the illustration 620 which shows a process of tumour extravasation of cancer cells (metastasis). The process includes tumour extravasation 624 of cells from circulation 622 to a metastasis site 626. At the metastasis site 626, the tumour goes through a premetastatic niche stage 628 then to micrometastasis 630. Cancer cells isolated from the biopsies are injected in a perusable vasculature network formed in a cell culture well insert in accordance with the present embodiments. These cancer cells' extravasation capabilities were then evaluated across the endothelial vasculature and to observe the formation of micrometastasis with a goal to assess how the extravasation and secondary tumour proliferation is affected by possible drug treatments.

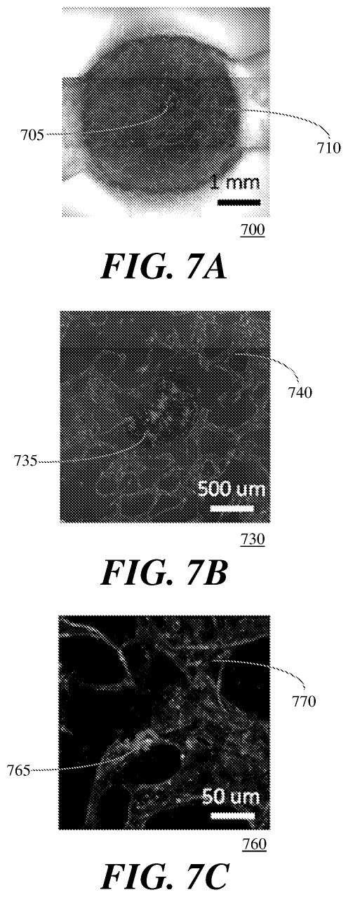

Referring to FIG. 7A, a fluorescent microscopy image 700 depicts a bottom view of the cell culture well insert in accordance with the present embodiments with a tumour tissue 705 (stained in red) surrounded by a vasculature network 710 (stained in green). FIG. 7B depicts a fluorescent microscopy image 730 of results of tests in accordance with the first aim shown in the illustration 600 and FIG. 7C depicts a fluorescent microscopy image 760 of results of tests in accordance with the second aim shown in the illustration 620. In the fluorescent microscopy images 730, 760, the tumour tissue 735, 765 (stained in red) is surrounded by a vasculature network 740, 770 (stained in green). The results in the fluorescent microscopy images 730, 760 of the two aims demonstrated the technology and protocols' readiness to be adapted to other tumour types, proving the cell culture well insert in accordance with the present embodiments as a competent personalized drug screening platform for oncology preclinical studies.

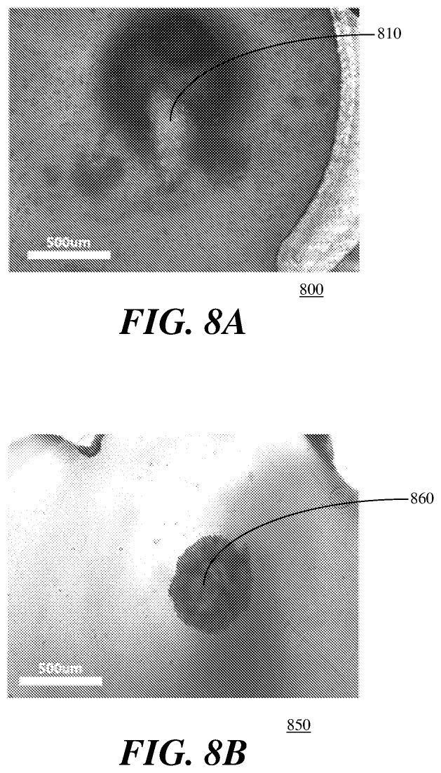

In accordance with the present embodiments, identified utilization protocols regarding the culture cell insert involve the usage of a hydrogel to support a 3D culture of cells. By confining the tissue/organoids 250 embedded in the hydrogel 262 in the central location of the cell culture well insert in accordance with the present embodiments without “leaking” on the lateral “half-moon” shaped channels 266, the cells 262 in the hydrogel 266 can be supported by nutrients or other soluble factors contained in the medium 264 as shown in FIG. 2G. The insert can be loaded with an empty hydrogel or with a hydrogel pre-loaded with cells. In the case of an empty hydrogel, cells can “invade” the hydrogel by colonizing it from the lateral liquid channel, if cells are injected into the culture media. When the cell culture insert is used to culture organoid/spheroids, and a user wants to obtain a quick vasculature network surrounding it, the most efficient identified protocol is to mix the organoids/aggregates with endothelial cells (EC) fibroblast. The mix of those two cells will allow the self-organization of a perfusable vasculature network. FIGS. 8A and 8B, depict fluorescence microscopy images 800, 850 of an example of 3D cell aggregate culture using hepatocellular carcinoma cell line (HepG2) aggregates expressing green fluorescent protein (GFP) surrounded by a Collagen type 1 hydrogel, where the image 800 depicts the hepatocellular carcinoma cell aggregates with embedded endothelial cells 810 (shown in red) and the image 850 depicts the hepatocellular carcinoma cell aggregates without embedded endothelial cells but with a necrotic core 860 typical for this type of tumour spheroid.

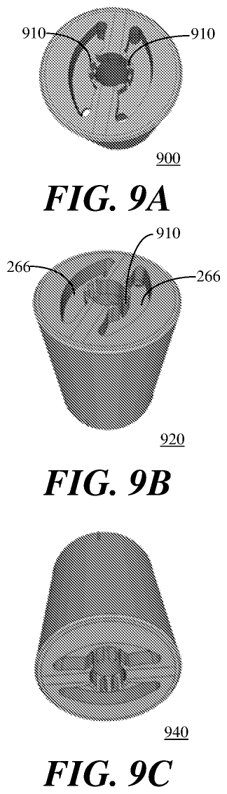

A “micro-net” design feature includes a mechanical obstruction to avoid the sample in an inner central chamber moving to one of the two side chambers. Basically, the design feature confines the tissue/organoids 250 embedded in the hydrogel 262 in the central location of the insert without “leaking” on the lateral “half-moon” shaped channels. Referring to FIGS. 9A, 9B and 9C, views 900, 920, 940 depict a “pillar” design which includes several pillars 910 arranged within the inflow and outflow from the inner central chamber having the hydrogel 262 to the channels 266.

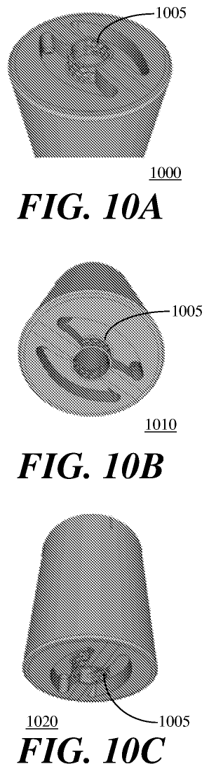

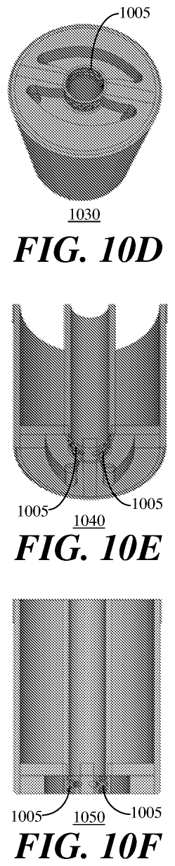

FIGS. 10A to 10F depict illustrations 1000, 1010, 1020, 1030, 1040, 1050 of details of a “net” design for a cell culture well insert in accordance with the present embodiments. The illustrations 1000, 1010 depict bottom view angles of the cell culture well insert having a net 1005 structure. It is to be noted that the net 1005 is not a “filter”; instead, the net 1005 structure of the “net” design, like the pillars 910 of the “pillar” design, are mechanical obstructions to avoid the sample in the inner central chamber from moving to the two side chambers.

The illustrations 1020, 1030 are bottom/side view angles of the cell culture well insert showing the net 1005 feature. The net 1005 feature may also be seen in the cross-section angled view 1040 and in the cross-section planar view illustration 1050. While the “net” design may be more challenging to produce with injection molding as compared to the “pillar” design, both designs are able to meet the primary requirement of confining a hydrogel in the center of the cell culture well insert without spilling into the side channel areas.

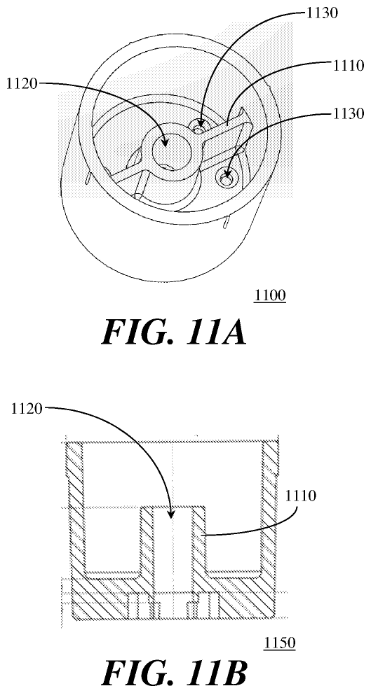

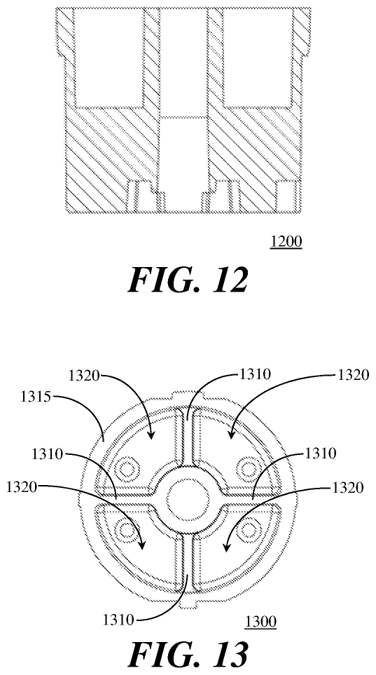

The “net” and “pillar” designs present some challenges in fabrication due to the small features. A half-wall design is another option tested to confine the hydrogel in the central region of the cell culture well insert in order to identify the best and easiest way to produce the cell culture well insert and the half-wall 1110 is an inner wall divider visible in a top/side view angle 1100 into the cell culture well insert in FIG. 11A and a cross-sectional planar view 1150 in FIG. 11B. In the top/side view angle 1100, a central hole 1120 is for the hydrogel injection and the smaller holes 1130 are for injection of the culture media. In addition to the half-wall design, alternatives to partially subdivide the top fluidic chamber of the cell culture well insert in accordance with the present embodiments include a half-deep design illustrated in a lateral cross-sectional planar view 1200 in FIG. 12 and a quarter-well design illustrated in a top planar view 1300 in FIG. 13. In the top planar view 1300, inner partitions 1310 at a same height as the outer wall 1315 subdivide the reservoir chamber into four chambers 1320. The reason for subdividing the reservoir chamber is to allow the fluid to flow from one hole to the other hole in the same chamber, translating to a fluidic flow in the longitudinal direction of the lateral channel. This fluidic flow allows a homogeneous distribution of cells during a lateral channel seeding, for example, or prevents an over aspiration of the media in a case of using a vacuum aspirator.

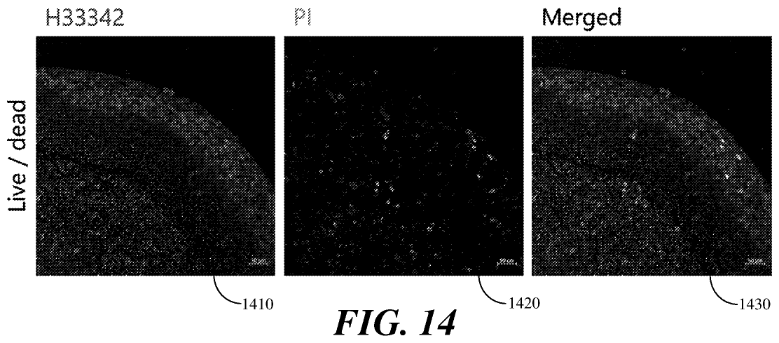

FIG. 14 depicts confocal microscopy images 1410, 1420, 1430 of in situ immunofluorescent staining of a liver organoid cultured in collagen hydrogel in a cell culture well insert in accordance with the present embodiments. The images 1410, 1420, 1430 have a 50 μm scale bar and depict live and dead staining of the liver organoid. The blue nuclear staining of live cells in the image 1410 and the red staining of dead cells in the image 1420 are merged in the image 1430.

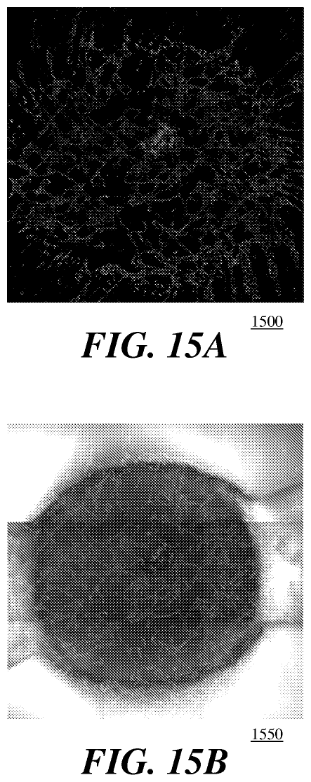

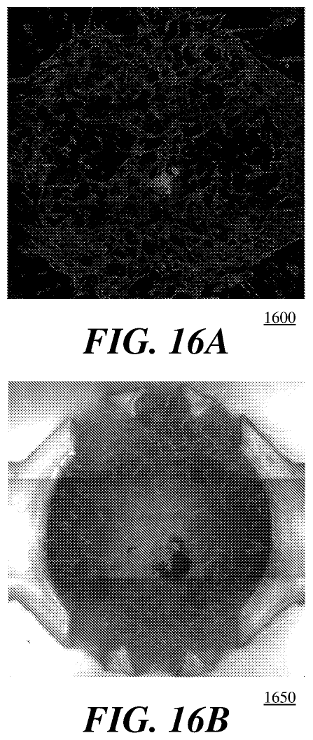

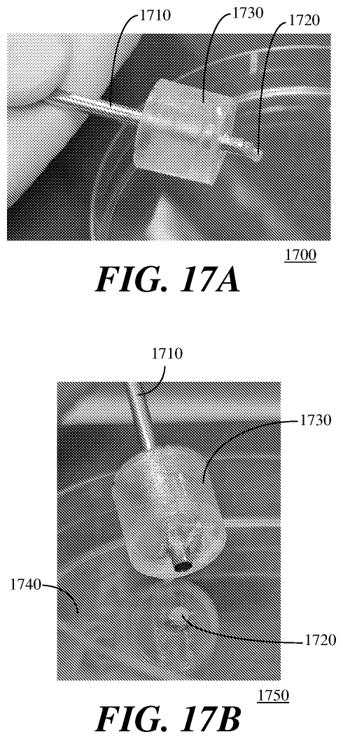

While the half-wall design and the “pillar” design are different structures, both designs are able to meet the primary requirement of confining a hydrogel in the center of the cell culture well insert without spilling into the side channel areas. In addition, as seen in FIGS. 15A and 16A and in FIGS. 15B and 16B, the results of tumor vascularization do not substantially differ when cultures in cell culture well inserts have the different designs. FIGS. 15A and 15B depict confocal microscopy images 1500, 1550 of vascularized tumors in a half-wall design of a cell culture well insert in accordance with the present embodiments and FIGS. 16A and 16B depict confocal microscopy images 1600, 1650 of vascularized tumors in a “pillar” design of a cell culture well insert in accordance with the present embodiments. The images 1500, 1600 depict results of tests in accordance with the vascularized process depicted in FIG. 6A and the images 1550, 1650 depict bottom views of the cell culture well insert including the results of the tests in accordance with the vascularized process.

Referring to FIGS. 17A and 17B, a process for removing a fresh (not frozen) sample from a cell culture well insert in accordance with the present embodiments is depicted in photographs 1700, 1750. In the photograph 1700, a rod 1710 pushes a sample 1720 (e.g., a hydrogel encased tissue/organoids) out of the cell culture well insert as the rod is passed into the volume formed by the inner wall 232 in a cell culture well insert 1730. The photograph 1700 depicts a stage in a sample retrieval process in accordance with the present embodiments immediately after the sample 1720 is pushed by the rod 1710 from the cell culture well insert 1730. The photograph 1750 depicts a later stage in the sample retrieval process where the sample 1720 is fully removed from the cell culture well insert 1730 and collected in a petri dish 1740.

The rod 1710 is a suitable cylindrical element having a diameter that matches the inner diameter of the hydrogel column (i.e., the volume defined by the inner wall 232 of the cell culture well insert 1730).

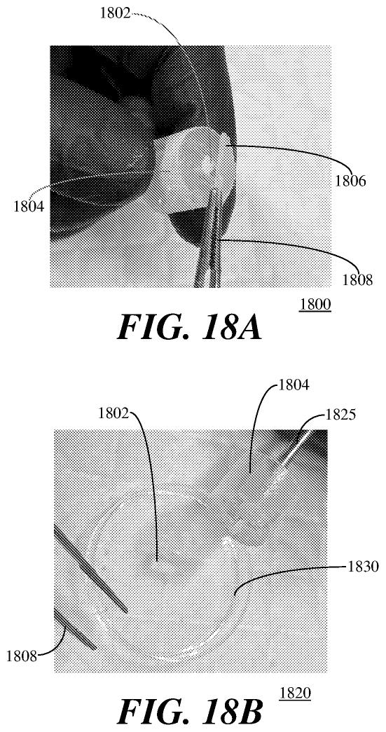

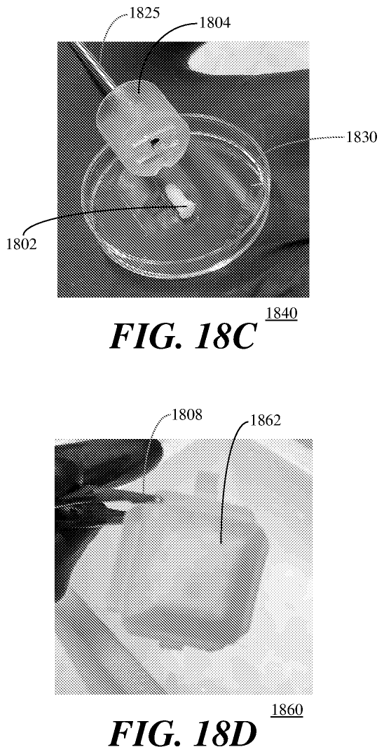

FIGS. 18A to 18D depict photographs 1800, 1820, 1840, 1860 illustrating stages in a process for removing a frozen sample from a cell culture well insert in accordance with the present embodiments. A sample 1802 can be rapidly frozen by exposing the cell culture well insert 1804 to a liquid nitrogen vapor. As shown in the photograph 1800, a membrane or laminate 1806 added to the bottom of the cell culture well insert 1804 can be easily peeled off with a pair of tweezers 1808. Using a bottom laminate 1806 advantageously enables use of the cell culture well insert 1804 in accordance with the present embodiments as a standalone 3D cell culturing device or in a dedicated holder or bottomless well plate.

Referring to the photographs 1820, 1840, a rod 1825 can enter from the top of the cell culture well insert 1804 and be used to push the frozen sample 1802 out from the cell culture well insert 1804 and into a petri dish 1830. The photograph 1860 depicts a later stage in the frozen sample retrieval process where the frozen sample 1802 is collected with the tweezers 1808 and added into a “cassette” 1862 for histology sample preparation.

Both described methods for collection of fresh samples and frozen samples can be used for further histological analysis and digital spatial profiling. While the native state fresh sample is preferred when live cells are required, freezing the sample before retrieval is preferred to minimize structural disruption during retrieval. Whether fresh sample collection or frozen sample collection is used, is driven by the downstream analysis/assay to be performed. Considering the tight fit of the insert in the wells of the multi-well cell culture plate, the tweezers 1808 may also be necessary to easily remove the insert from the well.

The cell culture cell inserts in accordance with the present embodiments advantageously enable cell culturing in a 3D cell culture matrix, providing solutions in many analytical technologies and assay types for testing patient-specific tumour sensitivity to anticancer compounds before clinical treatment administration and simplifying testing therapeutic strategies on patient-derived tumour organoids while retaining tumour complexity and allowing for rapid drug screening.

Thus, it can be seen that the present embodiments provide devices to enable testing patient-specific tumour sensitivity to anticancer compounds before clinical treatment administration thereby simplifying testing therapeutic strategies on patient-derived tumour organoids yet retaining tumour complexity and allowing for a rapid drug screening. The cell culture well insert in accordance with the present embodiments is a plastic insert fitting a standard 48-well cell culture plate. The insert can be easily mass-produced by injection moulding. Gradients can be generated across hydrogels culturing spheroids/organoids, the gradients being crucial to keeping spheroids/organoids viable and functional and for administering drugs or soluble factors for various screening applications. The design of the well insert allows oxygen exchange from the top (i.e., an open system) and the insert can be taken out from the well to retrieve the biological material.

The cell culture well inserts in accordance with the present embodiments can be used with existing multi-well plates (e.g., 24-or 48-well plates) with or without a bottom laminate or membrane. The size of the hydrogel compartment in the cell culture well inserts, for example (3 mm×1.5 mm(h)) for a 48-well culture plate, advantageously allows the culture of bigger organoids or tissue pieces in contrast to other microfluidic technologies. In addition, cultured cells/biopsies can be retrieved from the cell culture well inserts with their spatial organization intact for downstream analysis by cryosectioning, histology, digital spatial profiling, or other analytic processes.

The design of the cell culture well inserts in accordance with the present embodiments enables the gel plus organoid, biopsy, vasculature, or similar sample to be retained in the cell culture well insert's gel column (the inner cylindrical space formed by the inner wall 232). In addition, as there is more surface area in the column that is in contact with the gel than the well bottom/coverslip/laminate surface, the laminate can be easily removed without compromising the sample in the hydrogel.

Existing devices do not allow easy access to the gel, while the unlaminated cell culture well inserts in accordance with the present embodiments can advantageously be separated from a well of a multi-well plate without leaving the gel stuck on the well's bottom surface. A laminated device beneficially incorporates a feature that enables the easy removal of the laminate with the gel being retained in the central gel column, advantageously facilitating both a leak-proof culture and easy sample retrieval.

The gel can be pushed out using a suitably shaped implement such as the cylindrical rod 1710. 1825 having a diameter that matches the inner diameter of the gel column. Depending on the experimental requirements, the gel can be retrieved in its native state (if live cells are required, for example), or the gel can be frozen within the device before retrieval (to minimise any structural disruption during the retrieval process).

While exemplary embodiments have been presented in the foregoing detailed description of the present embodiments, it should be appreciated that a vast number of variations exist. It should further be appreciated that the exemplary embodiments are only examples, and are not intended to limit the scope, applicability, operation, or configuration of the invention in any way. Rather, the foregoing detailed description will provide those skilled in the art with a convenient road map for implementing exemplary embodiments of the invention, it being understood that various changes may be made in the function and arrangement of steps and method of operation described in the exemplary embodiments without departing from the scope of the invention as set forth in the appended claims.

1. A multi-well plate compatible cell culture device comprising:
an outer wall;
an inner wall within the outer wall and defining a volume within the inner wall, wherein the outer wall and the inner wall form a cavity therebetween;
a base connected to a bottom of the outer wall and a bottom of the inner wall and configured to position a cell culture sample below the volume within the inner wall; and
one or more partitions connected to the base and connecting the inner wall to the outer wall, wherein the one or more partitions segment the cavity into a plurality of voids,
wherein openings in surfaces forming the voids allow fluid flow from a first one of the voids and below the volume within the inner wall to a second one of the voids, and wherein the fluid flow is configured to interact with the cell culture sample when flowing through a sample region within the inner wall, wherein the sample region has a depth defined by a height of the inner wall greater than a length defined by an inner distance across the volume defined within the inner wall.

2. The cell culture well device in accordance with claim 1, wherein the outer wall is dimensioned to fit into a well of a cell culture dish or a cell culture plate.

3. The cell culture well device in accordance with claim 1, wherein the openings are formed in the base.

4. The cell culture well device in accordance with claim 1, wherein the openings are formed in the inner wall.

5. The cell culture well device in accordance with claim 1, wherein the openings comprise netted openings.

6. The cell culture well device in accordance with claim 5, wherein, in operation, the cell culture sample present in the fluid flowing from the first one of the voids and below the volume within the inner wall to the second one of the voids is retained as the fluid flows through the netted openings.

7. The cell culture well device in accordance with claim 1, wherein the openings comprise pillars.

8. The cell culture well device in accordance with claim 7, wherein, in operation, the cell culture sample present in the fluid flowing from the first one of the voids and below the volume within the inner wall to the second one of the voids is retained as the fluid flows through the openings comprise the pillars.

9. The cell culture well device in accordance with claim 1, wherein in operation the cell culture sample in enclosed in hydrogel, and wherein the base is configured to enclose the hydrogel within the sample region.

10. The cell culture well device in accordance with claim 1, wherein in operation, the fluid flowing from the first one of the voids and below the volume within the inner wall to the second one of the voids comprises a cell culture media.

11. The cell culture well device in accordance with claim 10, wherein in operation, the fluid flows from the first one of the voids and below the volume within the inner wall to the second one of the voids in response to a pressure gradient or pressure difference in volume of the fluid in the first one of the voids and volume of the fluid in the second one of the voids.

12. The cell culture well device in accordance with claim 1, wherein the openings comprise netted openings, and wherein in operation, the cell culture sample present in the fluid flowing from the first one of the voids and below the volume within the inner wall to the second one of the voids is retained as the fluid flows through the netted openings.

13. The cell culture well device in accordance with claim 1, wherein the cell culture sample is selected from the group consisting of organoids, spheroids, tissue, biopsy samples, and cell culture cell lines.

14. The cell culture well device in accordance with claim 1, wherein the partitions have a height less than a height of the outer wall.

15. The cell culture well device in accordance with claim 14 wherein the partitions have a height half the height of the outer wall.

16. The cell culture well device in accordance with claim 1, wherein the cavities formed between the inner wall and the outer wall are less deep than a height of the outer wall.

17. The cell culture well device in accordance with claim 1, wherein the one or more partitions connecting the inner wall to the outer wall comprise two or more partitions segmenting the cavity into two or more voids.

18. The cell culture well device in accordance with claim 17, wherein the two or more partitions connecting the inner wall to the outer wall comprise two partitions segmenting the cavity into two voids.

19. The cell culture well device in accordance with claim 17, wherein the two or more partitions connecting the inner wall to the outer wall comprise four partitions segmenting the cavity into four voids.

20. The cell culture well device in accordance with claim 1, further comprising a membrane or a laminate attached to an outer surface of the base for sealing the cell culture well insert.

21. Use of the cell culture well device in accordance with claim 1 as an insert into a well of a multi-well cell culture dish or a multi-well cell culture plate.

22. A cell culture device kit comprising:
a cell culture well device in accordance claim 1; and
a sample removal rod, wherein a shape of the sample removal rod is dimensioned to snugly fit within the volume defined within the inner wall of the cell culture well device.
